(19) United States
(12) Patent Application Publication
(10) Pub. No.: US 2018/0353323 A1
(43) Pub. Date: Dec. 13, 2018

(54) Composition Comprising a Sensory-Specific Material and Method of Manufacture

(71) Applicant: Charles Chung, San Francisco, CA (US)

(72) Inventor: Charles Chung, San Francisco, CA (US)

(21) Appl. No.: 15/757,829

(22) PCT Filed: Sep. 7, 2016
(86) PCT No.: PCT/US16/50560

§ 371 Date: Mar. 6, 2018

Related U.S. Application Data
(60) Provisional application No. 62/215,506, filed on Sep. 8, 2015.

(51) Int. Cl. A61F 6/04

(52) U.S. Cl. CPC A61F 6/04; A61F 2006/048

(57) ABSTRACT

A homogeneous material layer comprising a base layer and at least one effective layer is disclosed. The material layer is made of a first material characterized by a bulk modulus of elasticity. The effective layer is defined by a plurality of projections that project from the base layer. Each projection includes a stalk and a cap having an end face. The height-to-width aspect ratio of the stalks is selected such that the effective layer has an effective modulus of elasticity that is lower than the bulk modulus of elasticity. The arrangement of the projections is selected such that the end faces are spaced along at least one dimension by a spacing that is less than or equal to one millimeter, thereby controlling the fine roughness of the effective surface. Control of the effective modulus and the spacing of the end faces enables control of the feel of the effective layer.

### CROSS REFERENCE TO RELATED APPLICATIONS

This application claims the benefit of U.S. Provisional Application Ser. No. 62/215,506, filed Sep. 8, 2015, entitled “Composition Comprising a Sensory-Specific Material and Method of Manufacture” (Attorney Docket: 3080-002PR1), which is incorporated herein by reference. If there are any contradictions or inconsistencies in language between this application and one or more of the cases that have been incorporated by reference that might affect the interpretation of the claims in this case, the claims in this case should be interpreted to be consistent with the language in this case.

### FIELD OF THE INVENTION

The present invention relates to materials in general, and, more particularly, to structured-surface materials.

### BACKGROUND OF THE INVENTION

A material having realistic or enhanced touch sensitivity would be advantageous in many applications. For example, items such as gloves, surgical gloves, handles, containers (e.g., phone cases, etc.), condoms, and the like, would afford a user with significant benefits if the material of which they were composed provided a more realistic or tailored tactile response.

For example, a realistic sense of touch through surgical gloves could increase the likelihood of a surgeon detecting an abnormality during tissue palpation. A steering wheel having an enhanced-sensitivity surface could provide improved road feel to a driver. A condom having improved tactile characteristics would increase the likelihood of its use, which would have the benefit of reducing the transmission of venereal disease and the HIV virus.

Condoms, in particular, suffer from poor tactile performance. Most conventional condoms are formed from an impermeable elastomeric material that enables reasonable transmission of the sensations of pressure and temperature; however, few, if any other sensations are transmitted. Due to the reduced sexual sensation associated with most prior-art condoms, condom-use rates remain low—even though they provide nearly ideal contraception and HIV/STD protection.

In order to improve sensory transmission (e.g., sliding friction, etc.), the thickness of the material from which many conventional condoms, as well as surgical gloves, etc., are formed is reduced. Unfortunately, this can result in an increased risk of failure of the material, leading to increased potential for disease communication, unwanted pregnancy, contamination of sterile surgical fields, and the like.

The need for a material that provides good tactile characteristics without sacrificing material integrity and reliability remains, as yet, unmet in the prior art.

### SUMMARY OF THE INVENTION

The present invention reduces some of the disadvantages of the prior art by enabling improved sensory characteristics for a material without sacrificing mechanical integrity. Embodiments of the present invention are particularly well suited for use in applications where enhanced or high-fidelity tactile sensation is desirable, such as condoms, gloves, surgical gloves, and the like.

Embodiments of the present invention enable control over the “feel” of a material layer by structuring a surface region of the material layer to create an “effective layer” having an “effective surface.” The material layer is a homogeneous layer made of a first material that has a bulk modulus of elasticity. Because of its structure, however, the effective layer is characterized by an “effective modulus of elasticity” that is lower than the bulk modulus of elasticity. In addition, the surface structure of the effective layer defines the fine roughness of its effective surface. Collectively, the effective modulus of elasticity of the effective layer and the fine roughness of its effective layer determine the tactile characteristics of the structured region of the material layer. As a result, when in contact with tissue, the effective layer feels different than the same material would feel if it were unstructured. Effective layers in accordance with the present invention include straight walls, shaped walls, columnar-like projections, conical projections, hooked projections, and the like.

An illustrative embodiment of the present invention is a sheath layer of a condom comprising a layer of latex, where the sheath layer includes a base layer of substantially unstructured latex and an effective layer that is defined by a plurality of sub-millimeter-scale projections of latex that extend into the interior of the sheath layer. Each projection includes a stalk and a cap having an end face that is located at the free end of the stalk. The end faces collectively define an effective surface for the effective layer. The aspect ratio of the stalk is selected such that the effective layer is characterized by an effective modulus of elasticity that is lower than that of the unstructured latex base layer. Similarly, the fill factor of the end faces is selected to effect a desired fine roughness for the effective surface. The fine roughness and the effective modulus of elasticity can be independently controlled to achieve a desired feel for the effective layer, which gives rise to a broad latitude for the feel experienced by the condom wearer.

In some embodiments, the plurality of projections includes different projection types, including columns, cones, and walls of different heights, shapes, and stiffness, as well as projections having a non-linear shape, which are intermixed. In some embodiments, the projections are arranged in an arrangement comprising a plurality of fields, where each field selectively contains a different projection type.

In some embodiments, the projections are dimensioned and arranged to provide a specific desired tactile characteristic. In some of these embodiments, the desired tactile characteristic is the tactile characteristic of a naturally occurring surface.

In some embodiments, the projections include at least one projection type that is a “high aspect ratio” projection having ratio of height to width greater than 2:1.

In some embodiments, the projections include at least one projection type that is a “high-contrast projection” having an abrupt change in surface direction at the top and/or base of the projection.

In some embodiments, a layer of material includes projections on each major surface. In some embodiments, the projections on each major surface are dimensioned and arranged to interact with a specific type of tissue with which that surface is likely to come into contact. In some embodiments, the projections on each major surface are operative for mimicking the feel of a specific type of biological tissue, such as penile tissue, vaginal tissue, anal tissue, etc. In some of these embodiments, the projections on each major surface are operative for mimicking the feel of a different type of biological tissue.

In some embodiments, the projections are arranged in an arrangement that facilitates the storage of a fluid, such as silicone oil, within the arrangement. In some embodiments, the projections are dimensioned and arranged to trap and/or adsorb particulate, such as nanoparticles, included in the fluid.

In some embodiments, at least one projection includes a surface coating operative for controlling at least one surface characteristic. In some embodiments, at least one projection includes a surface coating that includes a plurality of features that are micron and/or nanometer scale.

An embodiment of the present invention is a condom comprising a layer (100) of a first material (114) characterized by a bulk modulus of elasticity, λ1, the layer including: a base layer (102) having a first surface (108) and a second surface (110); and a first plurality of projections (106) that project from the first surface, the first plurality of projections collectively defining a first effective layer (104), wherein each projection of the first plurality thereof includes a first end face (120) and a first stalk (116) having a first aspect ratio, and wherein the first end faces of the first plurality of projections collectively define a first effective surface (112); wherein the first effective layer is characterized by an effective modulus of elasticity, λ2, that is based on the first aspect ratio; wherein each first end face has a first width (d2) in at least one dimension, and wherein the first plurality of projections is arranged in a first arrangement in which the first end faces are spaced apart by a first spacing (s1) along the at least one dimension, and further wherein each of the first width and first spacing is less than or equal to 1 mm; and wherein λ2 is lower than λ1.

Another embodiment of the present invention is a method for forming a condom, the method comprising: (1) providing a first layer (100) such that it defines the condom sheath, wherein the first layer is provided such that it is homogeneous and comprises a first material (114) that is characterized by a bulk modulus of elasticity, λ1, the first layer comprising: (a) a base layer (102); and (b) a first effective layer (104) having a first effective modulus of elasticity, λ2, wherein the first effective layer includes a first effective surface (112) having a first fine roughness; (2) providing the first effective layer as a first plurality of projections (106) that project from a first surface (108) of the base layer, each projection of the first plurality thereof including: (a) a first stalk (116), wherein the first stalk is characterized by a first aspect ratio; and (b) a first cap (118) having a first end face (120) that has a first width (d2) in at least one dimension; wherein the first effective layer is provided such that the first plurality of projections is arranged in a first arrangement in which the first end faces are spaced apart by a first spacing (s1) along the at least one dimension; (3) selecting each of the first width and the first spacing such that it is less than or equal to 1 mm, wherein the first fine roughness is based on the first width and the first spacing; and (4) selecting the first aspect ratio such that λ2 is lower than λ1, wherein λ2 is based on the first aspect ratio.

Another embodiment of the present invention is a layer (100) of a first material (114) characterized by a bulk modulus of elasticity, λ1, the layer including: a base layer (102) having a first surface (108) and a second surface (110); and a first plurality of projections (106) that project from the first surface, the first plurality of projections collectively defining a first effective layer (104), wherein each projection of the first plurality thereof includes a first end face (120) and a first stalk (116) having a first aspect ratio, and wherein the first end faces of the first plurality of projections collectively define a first effective surface (112); wherein the first effective layer is characterized by an effective modulus of elasticity, λ2, that is based on the first aspect ratio; wherein each first end face has a first width (d2) in at least one dimension, and wherein the first plurality of projections is arranged in a first arrangement in which the first end faces are spaced apart by a first spacing (s1) along the at least one dimension, and further wherein each of the first width and first spacing is less than or equal to 1 mm; and wherein λ2 is lower than λ1.

Yet another embodiment of the present invention is a method comprising: (1) providing a first layer (100), wherein the first layer is provided such that it is homogeneous and comprises a first material (114) that is characterized by a bulk modulus of elasticity, λ1, the first layer comprising: (a) a base layer (102); and (b) a first effective layer (104) having a first effective modulus of elasticity, λ2, wherein the first effective layer includes a first effective surface (112) having a first fine roughness; (2) providing the first effective layer as a first plurality of projections (106) that project from a first surface (108) of the base layer, each projection of the first plurality thereof including: (a) a first stalk (116), wherein the first stalk is characterized by a first aspect ratio; and (b) a first cap (118) having a first end face (120) that has a first width (d2) in at least one dimension; wherein the first effective layer is provided such that the first plurality of projections is arranged in a first arrangement in which the first end faces are spaced apart by a first spacing (s1) along the at least one dimension; (3) selecting each of the first width and the first spacing such that it is less than or equal to 1 mm, wherein the first fine roughness is based on the first width and the first spacing; and (4) selecting the first aspect ratio such that λ2 is lower than λ1, wherein λ2 is based on the first aspect ratio.

### BRIEF DESCRIPTION OF THE DRAWINGS

FIG. 1A depicts a cross-sectional view of a condom sheath layer having an engineered feel in accordance with an illustrative embodiment of the present invention.

FIG. 1B depicts a schematic drawing of an expanded view of a portion of layer 100.

FIG. 1C depicts a projection in a preferred deformation mode.

FIG. 1D depicts a schematic drawing of a cross-sectional view of a high-contrast projection in accordance with the present invention.

FIG. 1E depicts a schematic drawing of an alternative projection in accordance with the present invention.

FIG. 2 depicts operations of a method suitable for forming layer 100.

FIG. 3A depicts a schematic drawing of a system suitable for templating layer 100 as a condom.

FIG. 3B depicts a schematic drawing of an exploded view of a portion of form 302.

FIGS. 3C-D depict cross-sections of a portion of form 302 before and after, respectively, feature layer 308 is wrapped around mandrel 306.

FIGS. 3E and 3F depict schematic drawings of an alternative process for fabricating a form, before and after the formation of surface features 310, respectively, in accordance with the present invention.

FIG. 4 depicts operations of a sub-method suitable for forming feature layer 308 in accordance with the illustrative embodiment.

FIGS. 5A-C depict schematic drawings of cross-sections of nascent feature layer 308 at different points in its fabrication.

FIGS. 6A-B depict schematic drawings of cross-sectional and top views, respectively, of a portion of a sheath layer having engineered feels on both sides in accordance with a first alternative embodiment of the present invention.

FIG. 7 depicts operations of a method suitable for forming layer 600 as a condom.

FIG. 8 depicts a schematic drawing of a mold suitable for templating layer 600 as a condom.

FIG. 9 depicts a schematic drawing of a cross-sectional view of a layer having improved sensory characteristics in accordance with a second alternative embodiment of the present invention.

FIGS. 10A-B depict schematic drawings of top and cross-sectional views, respectively, of a layer having improved sensory characteristics in accordance with a third alternative embodiment of the present invention.

### DETAILED DESCRIPTION

The following terms are defined for use in this Specification, including the appended claims:
feel (also referred to as sensory characteristic) of a surface is defined as the sensory perception of the nature of the surface, which depends on the physical stimuli that surface provides to the skin, or other tissue, with which it is in contact. Examples of physical stimuli include, without limitation, normal forces, shear forces, stiction, and the like.
engineered feel is defined as the sensory perception of the nature of a surface that has been physically modified such that its feel differs from the feel of an unmodified surface comprising the material of which the engineered surface is composed, where the physical modification controls at least one tactile parameter of the surface, including friction, fine roughness, and modulus of elasticity.
effective layer is defined as a surface portion of a homogeneous structural layer of a first material, wherein the surface portion has been physically modified to define a plurality of projections, where each projection is characterized by an end face, and the plurality of end faces have a width and spacing along at least one dimension that is one millimeter or less, and where at least one of size, shape, aspect ratio, distribution, compliance, and sharpness of the projections is selected to provide a desired engineered feel for the effective layer this is different than that of an unmodified surface of the first material. It should be noted that a plurality of projections whose end faces have width or spacing greater than one millimeter does not define an effective layer since each surface feature would be sensed individually.
effective surface is defined as a surface defined by the end faces of a plurality of projections that define an effective layer. An effective surface is typically not a continuous surface and, therefore, is characterized by a fine roughness that affects the feel of the surface. Effective surfaces in accordance with the present invention can have end faces that are co-planar, are non-co-planar, are orientated at one or more angles with respect to the effective surface, include sub-projections, and/or collectively define the effective surface such that it is non-planar.
aspect ratio is defined as the ratio of feature height to a lateral dimension (e.g., width) of the feature.
high aspect-ratio projections are defined as projections having a support stalk whose height-to-width ratio that is greater than 2:1.
high-contrast projection is defined as a projection having an end face whose outer corners have a radius of curvature that is less than or equal to 250 microns.

### Human Tactile Perception

Although a comprehensive model of human touch perception is still developing, it is generally accepted that the feel of a material surface is dictated by a set of five parameters: fine roughness (surface feature having width and spacing ≤1 mm), coarse roughness (surface feature having at least one of width and spacing >1 mm), compliance (hardness/softness), friction, and temperature.

The friction of a surface is a function of several factors, the surface structure of both the surface and the tissue with which it is in contact, as well as lubrication (both natural and applied) at the interface of the surface and the tissue.

Typically, the temperature of a material is an extrinsic characteristic that is based on its surrounding environment. For example, material layers suitable for use in human-perception applications (e.g., condoms, surgical gloves, etc.) are normally very thin. As a result, their temperature is dictated by that of the surface and/or tissue with which they are in contact.

Fine roughness describes the characteristics of a surface based on surface features whose separation at the surface is so close that they are not sensed as individual features but, rather, as a continuous surface. The fingertips have the finest tactile discrimination of individual points in the human body, with a threshold for sensing surface structure (i.e., protrusions, depressions, etc.) of 1 mm, below which surface features are not individually sensed.

Coarse roughness describes the characteristics of a surface based on surface features whose separation can be individually sensed. At the fingertips, this threshold is at surface structure larger than 1 mm.

Compliance describes the softness (or hardness) of a material. Every material is characterized by a bulk “modulus of elasticity” (hereinafter referred to as “modulus”) that is indicative of its compliance. The modulus of a material is the inverse ratio of how much the material deforms in response to a compressive force. As a result, the units for compliance or modulus are pressure and the standard SI unit for pressure is the Pascal (Pa).

Control of the temperature of a layer in accordance with the present invention is difficult to control. In addition, controlling coarse roughness does not appreciably affect the feel of a layer, since widely spaced features are individually discernable and do not significantly contribute to the feel of the layer as a quasi-continuous layer. It is an aspect of the present invention, however, that a desired feel of a surface can be attained by engineering the fine roughness of the surface and the modulus of the layer comprising the surface. In some cases, the desired feel can be further controlled by including friction-inducing elements at the surface.

### Principle of the Invention

The present invention enables the feel of at least one surface of a homogeneous material layer to be engineered such that it has a feel that differs from that of the material that composes the layer. The desired feel for the engineered surface is achieved by creating an effective layer and controlling both the modulus of the effective layer and the fine roughness and friction of its effective surface. Specifically, the original surface of the material layer is modified to create a plurality of projections that extend from an unmodified portion of the material layer (i.e., a base layer) to collectively define the effective layer. The end faces of the projections collectively define an effective surface that replaces the original surface of the material layer.

Each projection includes a stalk and a cap having an end face, where the aspect ratio of the stalks substantially dictates an effective modulus of the effective layer. The fine roughness of the effective surface is dictated by the size of the end faces and the spacing between them, each of which is kept at or below one millimeter so that the effective surface is seemingly continuous.

The desired feel for the effective layer is achieved by judicious selection of the aspect ratio of the stalk and the fill factor of the end faces (i.e., the size of the end faces and the spacing between them) to give rise to a modulus for the effective layer and a fine roughness for the effective surface in accordance with the desired feel of the engineered surface of the material layer. In some embodiments, the feel of the engineered surface is further controlled by providing sharp features at the end faces, thereby altering its fine roughness and friction parameters.

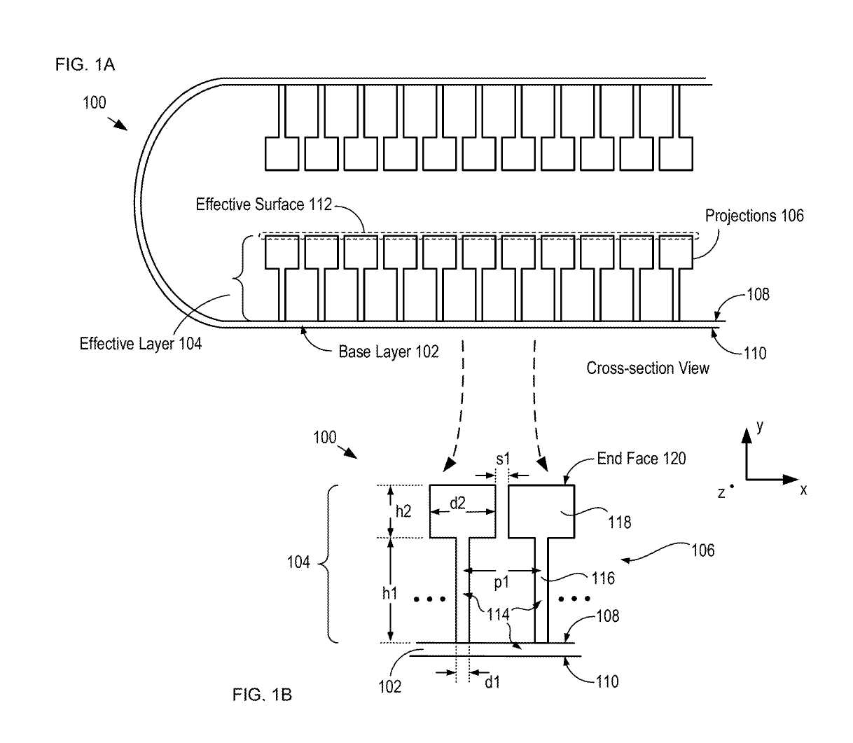

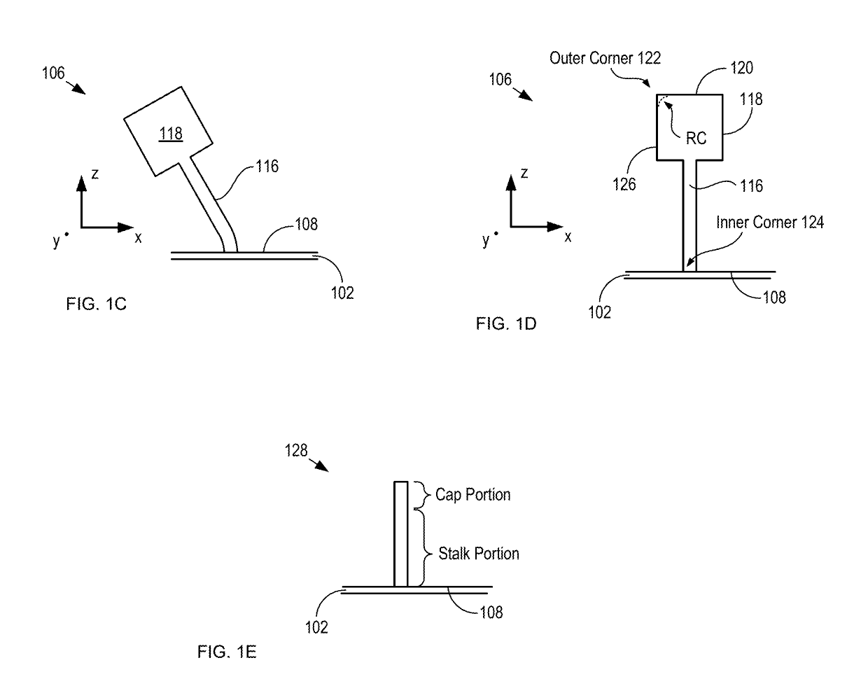

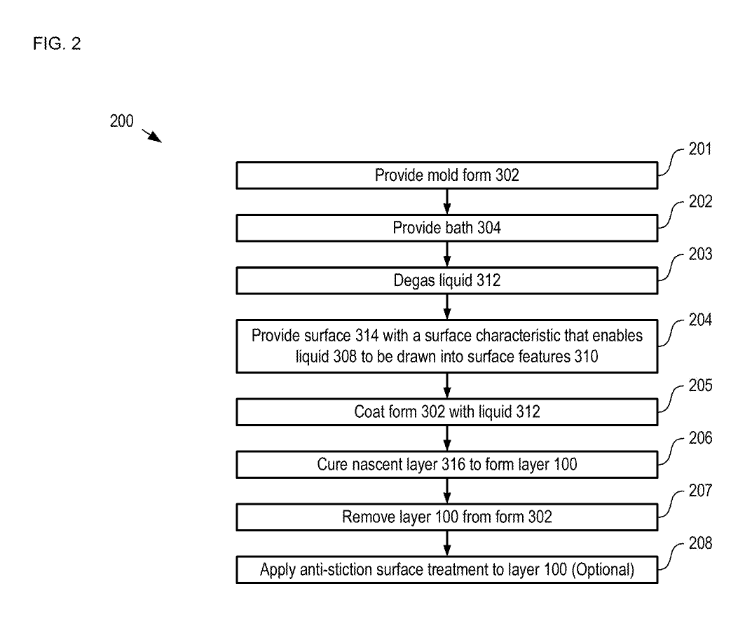

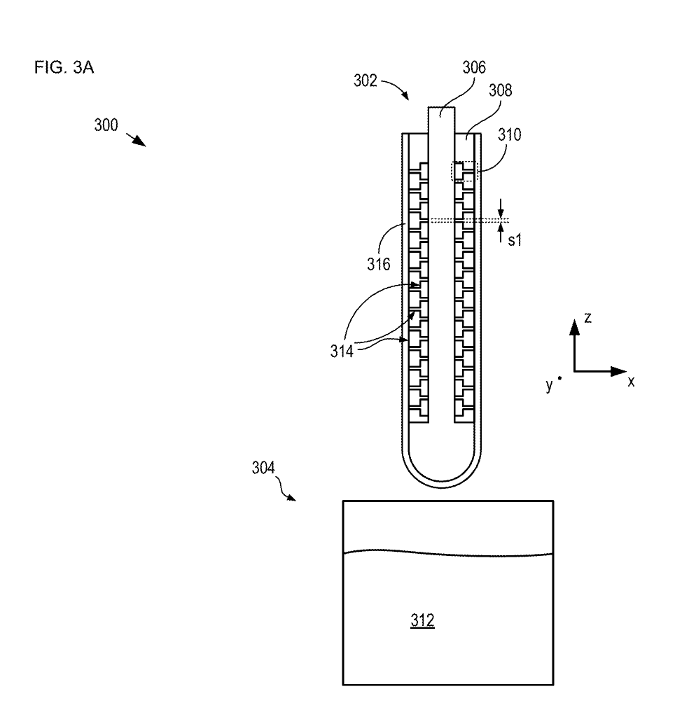

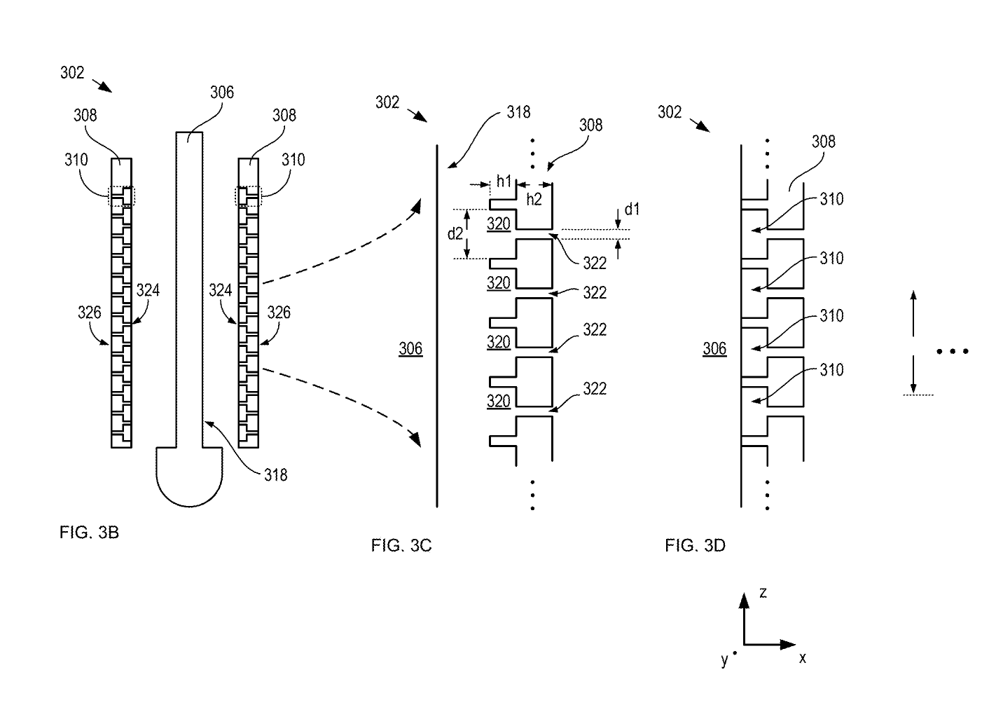

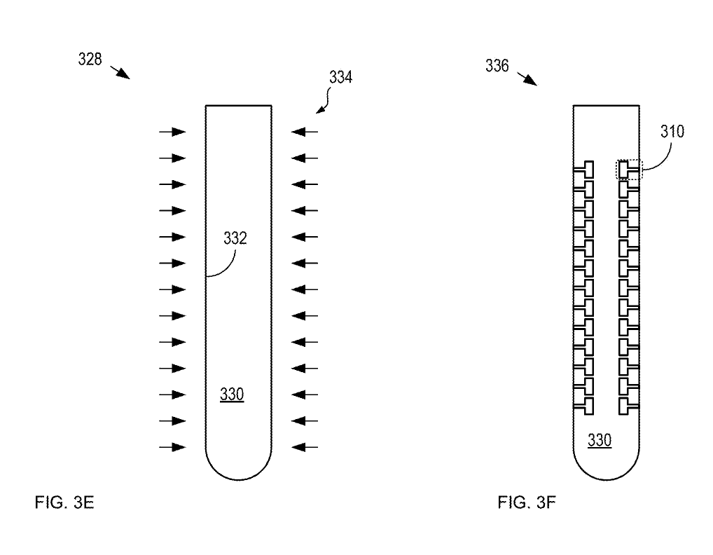

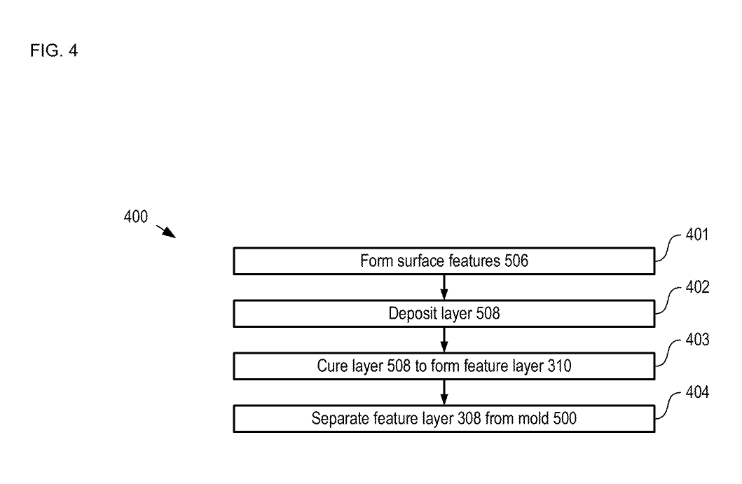

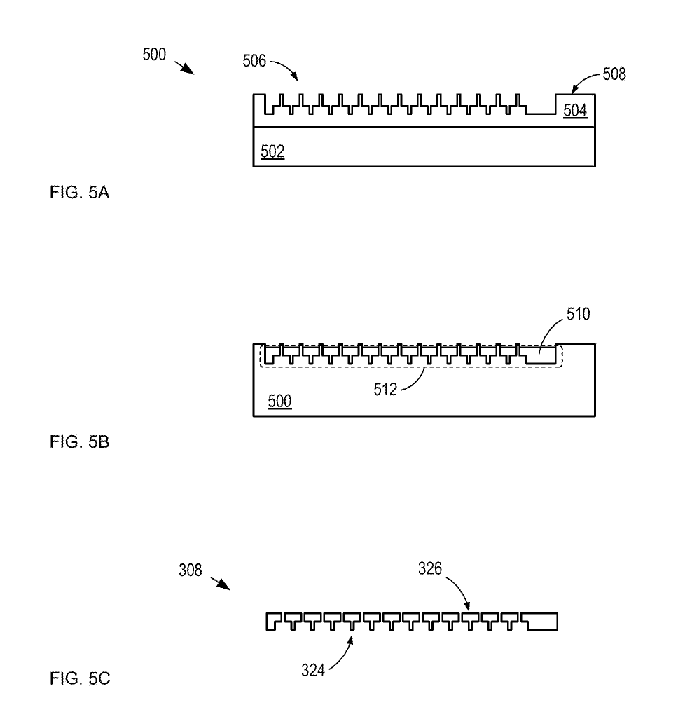

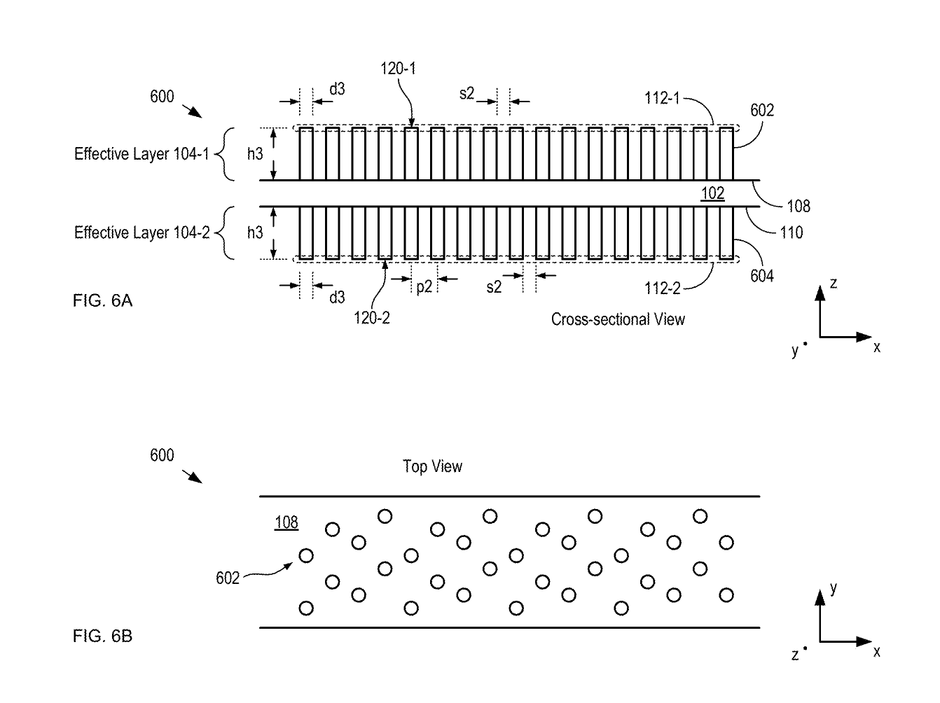

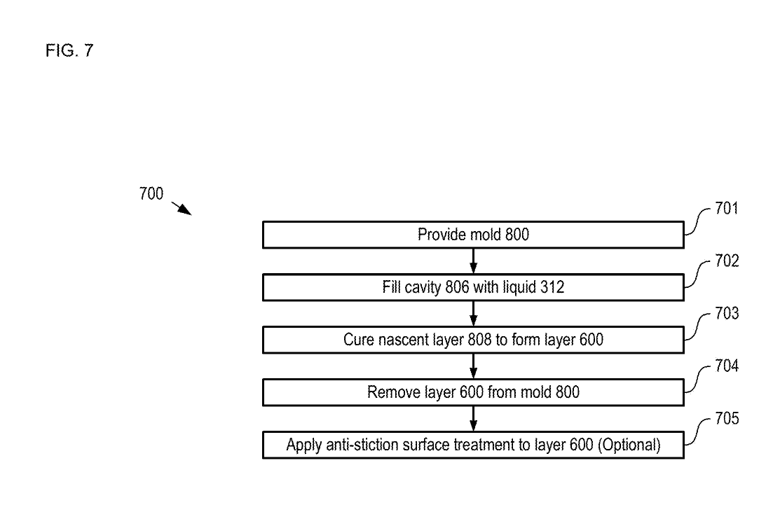

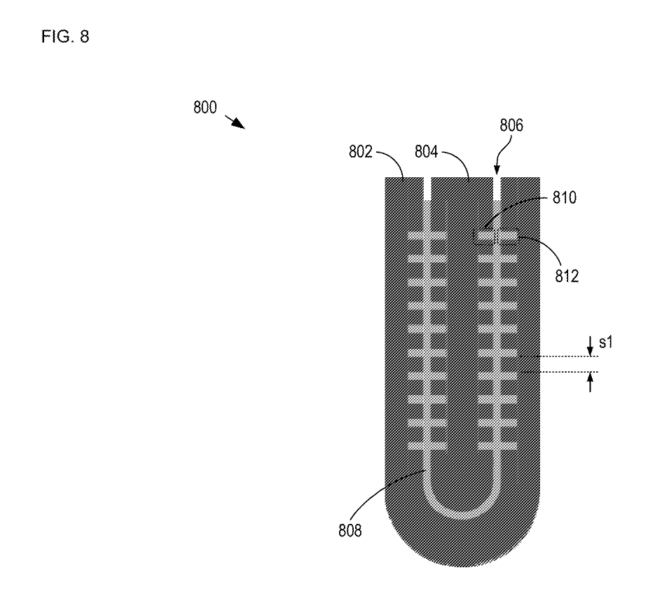

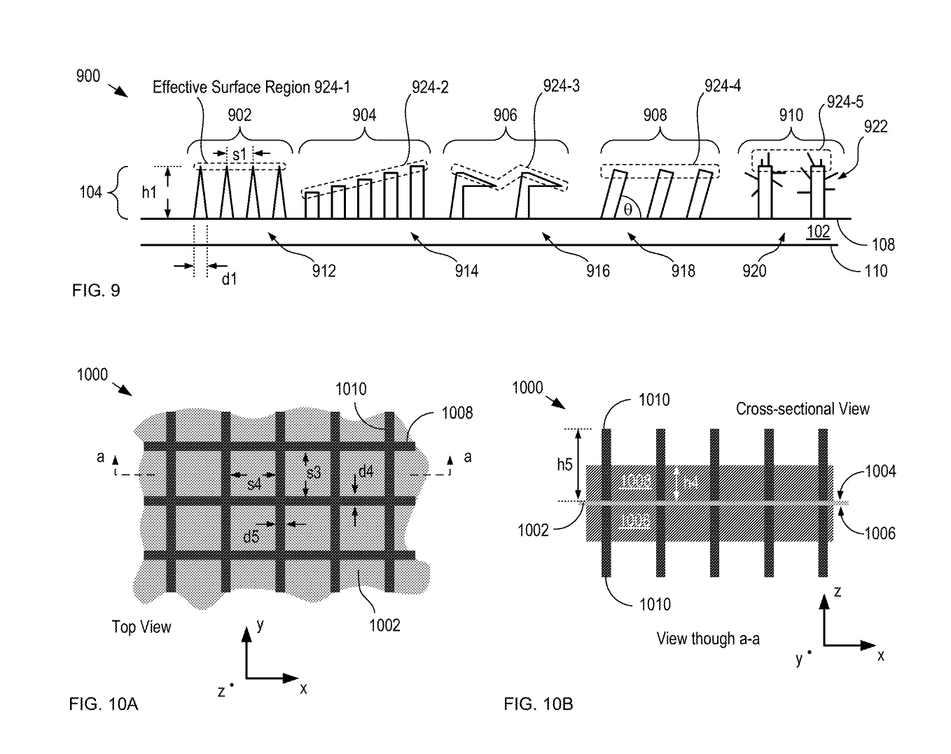

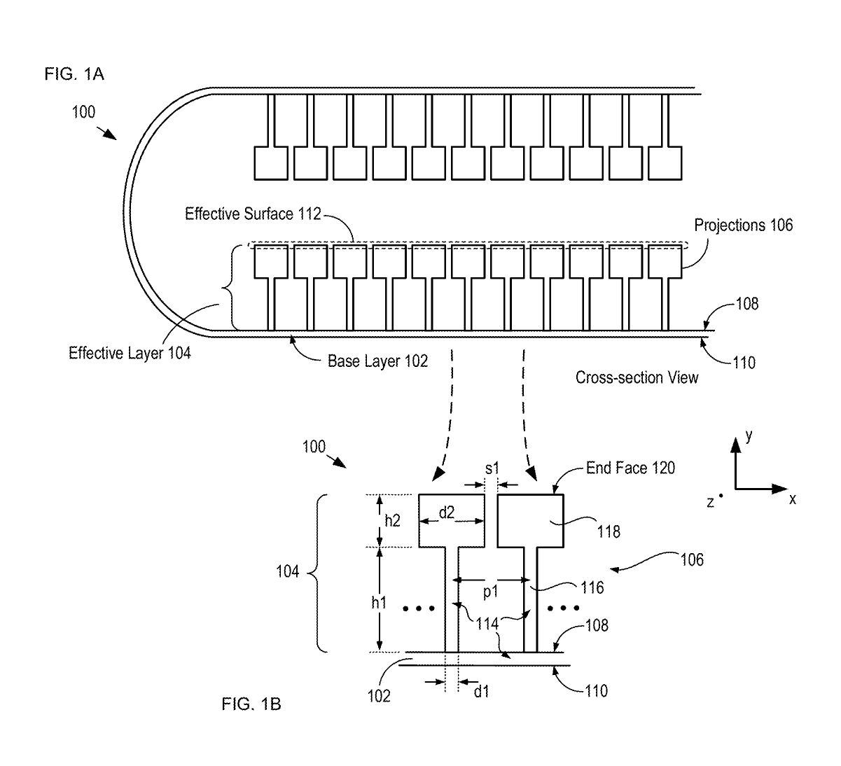

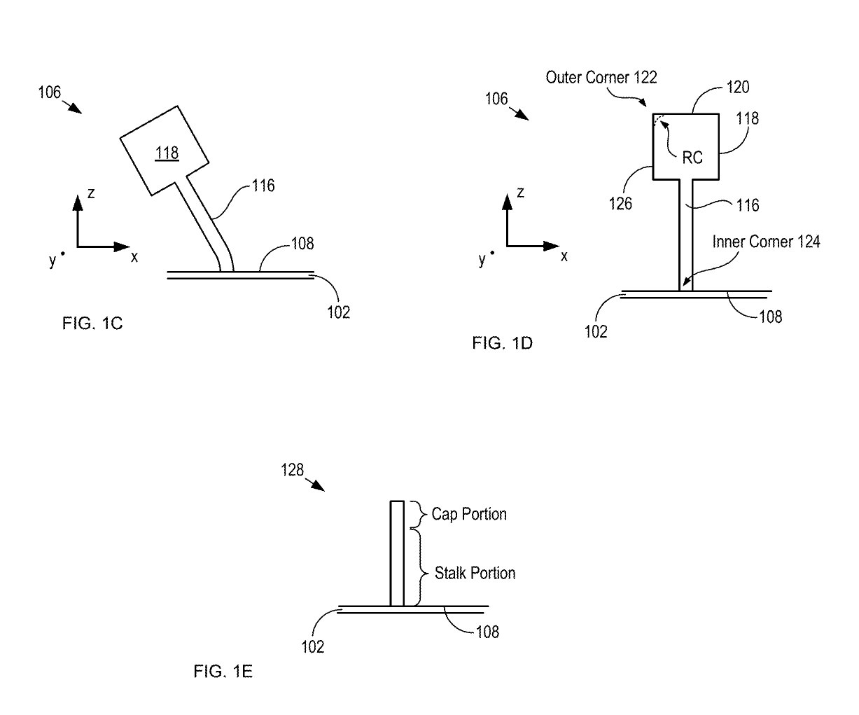

FIG. 1A depicts a cross-sectional view of a condom sheath layer having an engineered feel in accordance with an illustrative embodiment of the present invention. Layer 100 is a homogeneous layer that includes base layer 102 and effective layer 104, each of which is made of material 114. In the depicted example, material 114 is latex having a bulk modulus, λ1, of about 10 MPa. Layer 100 is formed to define a condom sheath. Although the illustrative embodiment comprises a layer formed to define a condom sheath, it will be clear to one skilled in the art, after reading this Specification, how to specify, make, and use alternative embodiments of the present invention wherein layer 100 is formed to define other articles, such as a surgical gloves, garments, and the like.

Although material 114 is latex in the illustrative embodiment, it will be clear to one skilled in the art, after reading this Specification, that material 114 can comprise a wide range of different materials without departing from the scope of the present invention, and that the choice of material 114 is typically based upon the application for which layer 100 is intended. Materials suitable for use in embodiments of the present invention include, without limitation, latex, polyurethane, silicone, rubber, silicone rubber, urethanes, AT-10 resin, polyisoprene, elastomers, and polymers, among others.

Base layer 102 is a layer having surfaces 108 and 110. Base layer 102 has a thickness, t1, of approximately 50 microns. In some embodiments, t1 has another value within the range of approximately 10 microns to approximately 1 millimeter and, preferably, within the range of approximately 15 microns to approximately 150 microns. One skilled in the art will recognize, after reading this Specification, that the thickness of base layer 102 is a matter of design choice based on the application for which an embodiment of the present invention is intended. As a result, any practical thickness for base layer 102 can be used without departing from the scope of the present invention.

Effective layer 104 is a portion of layer 100 that is shaped to define surface features (i.e., projections 106), each of which also comprises material 114. As discussed below, by virtue of the composite structure of projections 106, effective layer 104 has an effective modulus that is significantly lower than that of base layer 102.

Projections 106 are stand-alone walls that project normally from surface 108 and fully encircle the interior surface of the condom sheath. In the depicted example, projections 106 are substantially parallel “T-shaped” walls that are aligned with the y-direction and are periodically arranged along the x-direction with a period, p1, of 600 microns. It should be noted that the value of p1 can also affect the effective modulus of effective layer 104 and that the value of p1 can be selected as substantially any practical value without departing from the scope of the present invention. In some embodiments, effective layer 104 includes projections that are arranged in an aperiodic arrangement.

FIG. 1B depicts a schematic drawing of an expanded view of a portion of layer 100. Each of projections 106 includes stalk 116 and cap 118, which includes end face 120. End faces 120 collectively define effective surface 112 of effective layer 104.

Stalk 116 has a substantially uniform width, d1, of 100 microns and a height, h1, of 800 microns. As a result, the height-to-width aspect ratio of support 116 is 8:1. An 8:1 aspect ratio for stalk 116 gives rise to an effective modulus, λ2, for effective layer 104 that is significantly lower than the bulk modulus, λ1, of material 114 and, therefore, provides a more compliant feel for the condom wearer.

Projections 106 are high aspect-ratio projections in that the aspect ratio of stalk 116 is greater than 2:1. This facilitates buckling and bending that is substantially concentrated in the stalk in response to an applied force. Although in the illustrative embodiment, the aspect ratio of stalk 116 is 8:1, in some embodiments, the aspect ratio of the stalk is another ratio within the range of approximately 2:1 to approximately 40:1. Preferably, projections 106 are dimensioned and arranged such that stalk 116 deforms under an external load by bending and, in some cases, buckling. As a result, the preferred aspect ratio for stalk 116 is typically within the range of approximately 5:1 to 20:1 for most materials.

Cap 118 is the free end of projection 106. Cap 118 has a substantially uniform height, h2, and width, d2, each of which is approximately 500 microns. Due to their low aspect ratio, caps 118 are substantially inflexible and don't contribute significantly to the effective modulus for effective layer 104.

End faces 120 are separated by spacing, s1, which defines the fine roughness of effective surface 112. In the depicted example, end faces are spaced apart by an s1 that is equal to 100 microns. Effective surface 112, therefore, has a fill factor of approximately 83% in the x-direction, which provides a surface feel that is both stimulating and relatively “soft.” The value of each of d2 and s1 is less than or equal to approximately one millimeter, above which, surface features can be individually sensed. As discussed above, by limiting these dimensions to one millimeter or less, effective surface 112 feels like a substantially continuous surface to the user.

In some embodiments, d2 and s1 have different values that give rise to a different fill factor, along at least one lateral dimension, within the range of approximately 0.0001% and approximately 99%—and, preferably, within the range of approximately 0.04% to approximately 72%. For effective layers having uniform d2 and s1, the effective layer is characterized by a single spatial frequency. In embodiments wherein at least one of d2 and s1 are non-uniform, the effective layer is characterized by a broad range of spatial frequencies that are not centered on any particular spatial frequency.

It should be noted that, since the values of h1, d1, h2, d2, p1, and s1 are individually selectable, each can be independently controlled as necessary to obtain the desired feel for effective layer 104. In some embodiments, at least one of d1 and d2 is another width within the range of approximately 10 nanometers to approximately 1 millimeter. In some embodiments, d1 and d2 are substantially equal. In some embodiments, at least one of h1 and h2 has a different value within the range of approximately 10 nanometers to several centimeters. One skilled in the art will recognize, however, that the dimensions of one or more projections can be within any practical range based upon the application for which an embodiment is intended.

In some embodiments, caps 118 have a different aspect ratio. In some embodiments, caps 118 have a different cross-sectional shape, such as trapezoidal, etc., wherein their width is not uniform through their thickness.

FIG. 1C depicts a projection in a preferred deformation mode. Under load, projection 122 develops a restoring force that imparts a resistive force on the tissue with which it is in contact, thereby inducing a sensory response in that tissue.

In some embodiments, projections 106 have a non-uniform cross-sectional area that facilitates their being self-supporting and more inclined to buckling under load. For example, in some embodiments, projections 106 have a wider base than top. In some embodiments, projections 106 have a wider top than base. In some embodiments, projections 106 have a width that mitigates their touching each other during formation and/or until fully cured.

It should be noted that spacings within the ranges described above have been found to afford embodiments of the present invention particular advantage over the prior art in that it enables the emulation of particularly desirable materials (e.g., velvet, silk, fur, corduroy, etc.).

Table 1 below summarizes experimental results for the feel of different layers comprising projections 122 with different dimensions and arrangements.

TABLE 1

| | | Width of | Height of | | Fill | Modulus |
|---|---|---|---|---|---|---|
| Effective | Space | feature | feature | Aspect | Factor | Reduction |
| Feel | [um] | [um] | [um] | Ratio | [%] | Factor |
| “Soft” | 500 | 100 | 800 | 8 | 17% | 1868× |
| “Velvety” | 1000 | 100 | 800 | 8 | 9% | 3424× |
| “Corduroy” | 1000 | 150 | 800 | 5.3 | 13% | 1061× |

The effective feel was determined for latex test sample layers whose effective layers 104 comprise different arrangements of substantially identical projections, where the projections were free-standing walls arranged with uniform pitch. It clear that a wide range of sensory characteristics for layer 100 can be achieved by varying the effective modulus of effective layer 104 and the fine roughness of effective surface 112 in accordance with the present invention. For example, projections having a relatively higher aspect ratio and relatively smaller spacing provide a surface with a “softer” feel, while projections having a relatively lower aspect ratio provide a surface with a “harder” feel.

It is yet another aspect of the present invention that the “sharpness” of projections 106 also impact the feel of effective layer 104. For example, projections having “sharp” tips and/or corners represents a fine roughness and friction that forces pressure into a smaller area, thereby creating a concentrated, intense point of stimulation. Projections having “dull” tips and/or corners do not focus imparted forces into a small area and, therefore, create less intense stimulation. In the prior art, for example, bumps, studs, and ribs are often included on a condom surface; however, these conventional surface features have radii of curvature that are 500 microns or greater. As a result, such prior-art condoms are generally poorly received and do not foster widespread use.

FIG. 1D depicts a schematic drawing of a cross-sectional view of a high-contrast projection in accordance with the present invention. Projection 106 comprises outer corners 122 and inner corners 124. Outer corners 122 have radii of curvature RC and are formed by the intersection of sidewalls 126 and end face 120. In the depicted example, RC is 250 microns.

Because projections 106 are both high-aspect-ratio projections and high-contrast projections, they give rise to a surface that simultaneously feels soft and, yet, is highly stimulating.

In addition to providing a specific feel for a surface (or surfaces) of base layer 102, in some embodiments, at least some of projections 106 are arranged to facilitate holding a fluid, such as silicone oil, etc., within effective layer 104 and on outer surface 108. One skilled in the art will recognize, after reading this Specification, that the manner in which projections 106 are arranged for this purpose is a design parameter that is based on many factors, such as contact angle of material 114, surface tension and viscosity of the fluid, characteristics of particulates in the fluid, aspect ratio of projections 106, shape of the projections, and the like.

In some embodiments, projections 106 are formed with a relatively larger surface to improve their ability to collectively hold a fluid. In some embodiments, the projections are dimensioned and arranged to trap and/or adsorb matter, such as nanoparticles, lubricants, etc., included in a fluid. One skilled in the art will recognize, after reading this Specification, that matter (e.g., particulates, emollients, nanoparticles, etc.) included in a fluid trapped by projections 106 can be used to tailor the surface properties of the projections and, therefore, effective layer 104-1.

Although in the illustrative embodiment projections 106 are T-shaped, in some embodiments of the present invention, at least one of projections 106 has a different shape.

FIG. 1E depicts a schematic drawing of an alternative projection in accordance with the present invention. Projection 128 is analogous to projection 106; however projection 128 has the shape of a straight wall. In other words, in projection 128, cap 118 has the same cross-sectional shape as stalk 116 (i.e., the cap is simply the end portion of the stalk). In such embodiments, the thickness of the cap region contributes to the aspect ratio of the stalk and, therefore, bending and buckling occurs throughout the entire length of the projection.

In some embodiments, at least one of projections 106 has a shape that is non-linear, such as curved, wavy, polygonal, etc. In some embodiments, at least some of projections 106 are individual projections (e.g., columns and the like) rather than continuous walls. In some embodiments, at least some of projections 106 are lines and/or blocks (i.e., their lateral dimensions are unequal). In some embodiments, at least some of projections 106 have different heights. In some embodiments, at least one of projections 106 projects from surface 108 at an angle of other than 90°. In some embodiments, projections 106 extend from both surfaces 108 and 110.

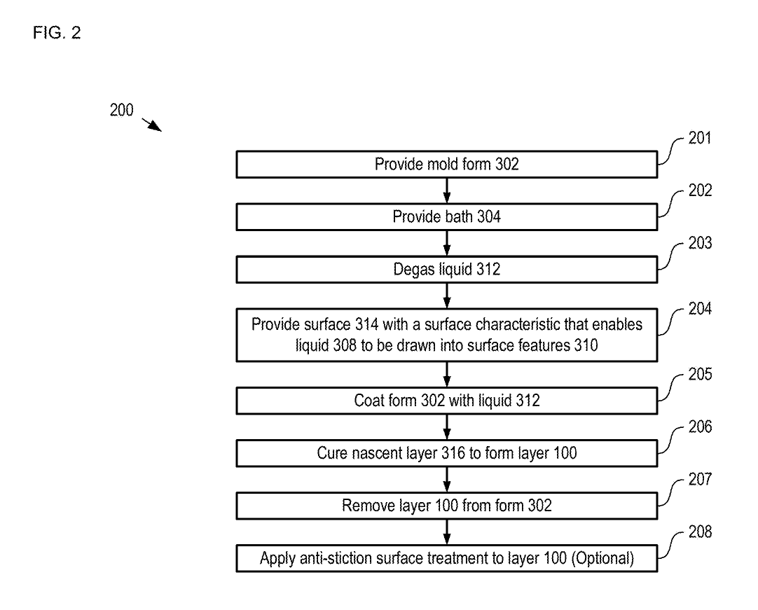

FIG. 2 depicts operations of a method suitable for forming layer 100.

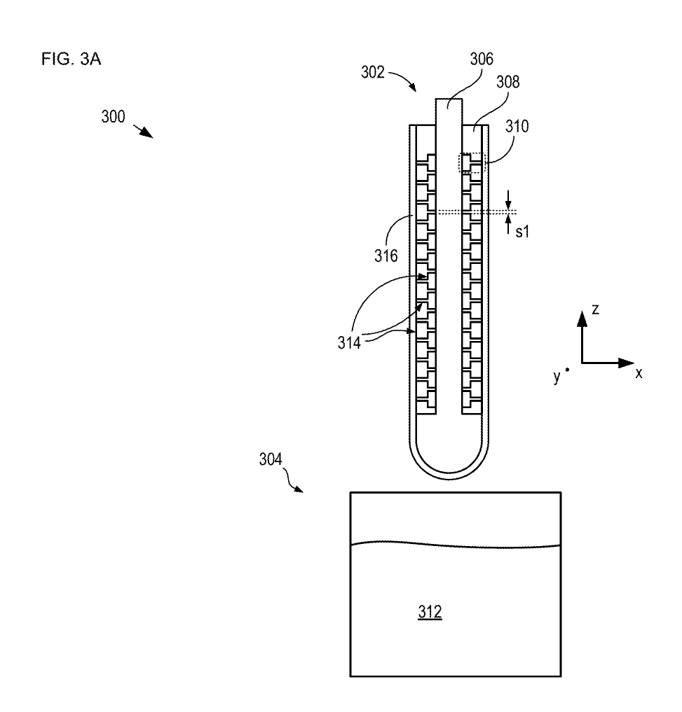

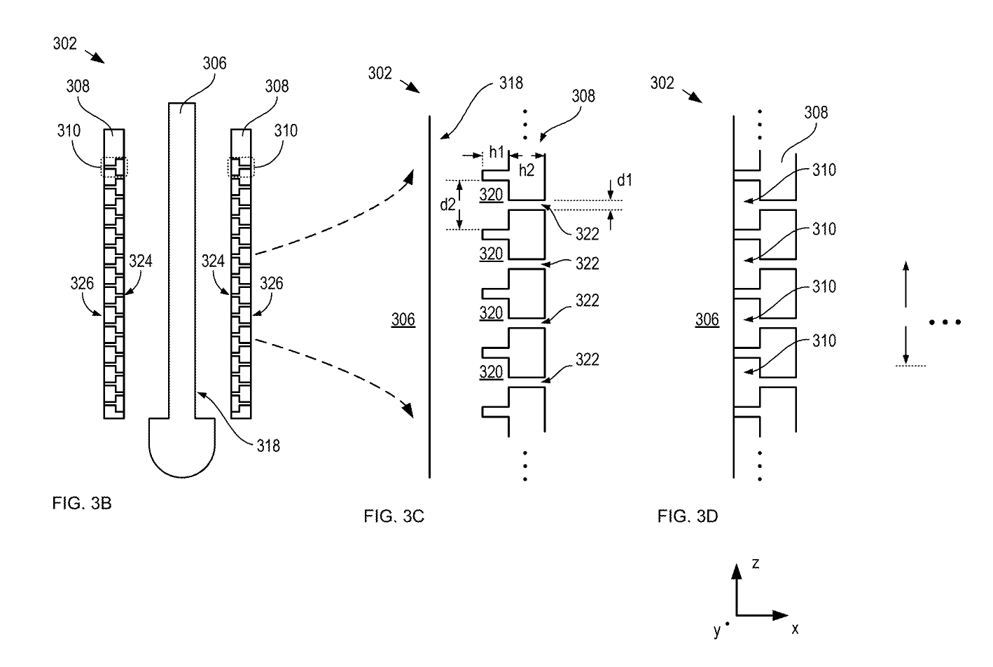

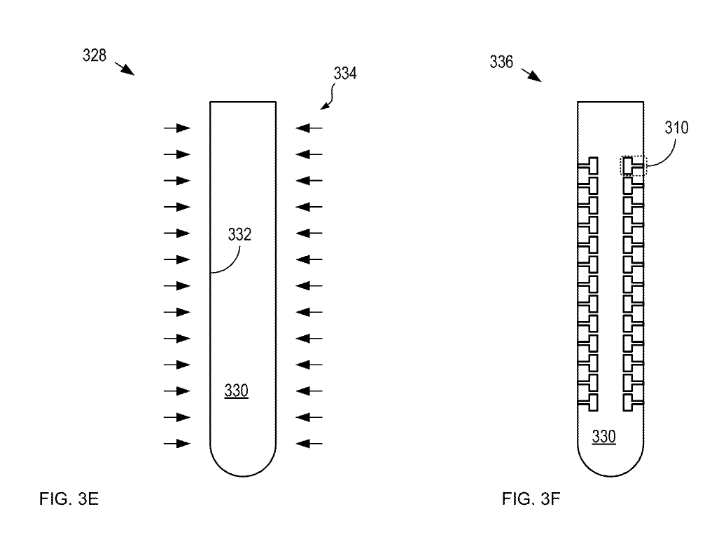

FIG. 3A depicts a schematic drawing of a system suitable for templating layer 100 as a condom. FIG. 3A depicts the formation of layer 100 while the layer is in its nascent state. System 300 includes form 302 and bath 304.

Method 200 is described with reference to FIGS. 1A-E and FIGS. 3A-D. Method 200 begins with operation 201, wherein mold form 302 is provided.

FIG. 3B depicts a schematic drawing of an exploded view of a portion of form 302. Form 302 includes mandrel 306 and feature layer 308, which is wrapped around the mandrel to provide form 302 with an outer surface that includes features suitable for defining projections 106. Feature layer 308 includes wells 320 and channels 322, which collectively define surface features 310.

Mandrel 306 is a rod of glass, metal, ceramic, or other material having surface that is relieved for receiving feature layer 308 such that, when the feature layer is in place, the outer surface of the feature layer is flush with the outermost surface of the mandrel.

Feature layer 308 is a layer of polydimethylsiloxane (PMDS), polymer, metal, glass, ceramic, or other material that includes features 310.

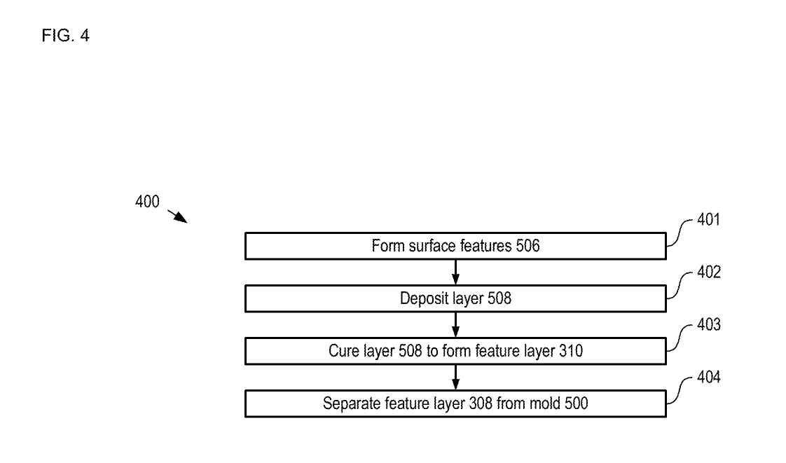

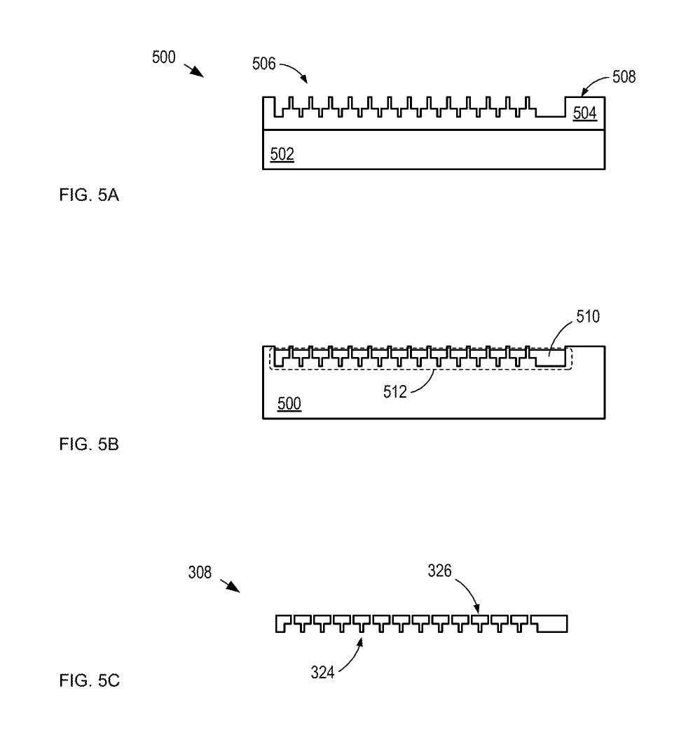

FIG. 4 depicts operations of a sub-method suitable for forming feature layer 308 in accordance with the illustrative embodiment. Sub-method 400 is described with continuing reference to FIGS. 3A-D, as well as reference to FIGS. 5A-C.

FIGS. 5A-C depict schematic drawings of cross-sections of nascent feature layer 308 at different points in its fabrication.

Sub-method 400 begins with sub-operation 401, wherein mold 500 is formed. Mold 500 includes surface features 506, which are formed in surface 508 of layer 504, which is disposed on substrate 502.

Substrate 502 is a conventional substrate suitable for planar processing.

Layer 504 is a layer of a material that supports the formation of micron- and sub-micron-scale features in its surface 508. In the depicted example, layer 504 comprises a photodefinable epoxy-based photoresist, such as a bisphenol phenol-formaldehyde resin-based epoxy (e.g., SU-8); however, one skilled in the art will recognize that many alternative materials can be used for layer 504. In some embodiments, layer 504 is not included and surface 508 is the top surface of substrate 502.

Surface features 506 are photodefined in layer 504 such that they have the inverse shape of wells 320 and channels 322. In some embodiments, surface features 506 are formed in another conventional manner, such as via a series of etch steps that define their shape. In some embodiments, surface features 506 are formed via conventional surface micromachining processes including, in some cases, the use of sacrificial layers. In some embodiments, surface features 506 are formed via nanoimprinting.

FIG. 5A depicts mold 500.

At operation 402, layer 510 is cast into mold 500 to define nascent feature layer 512. In the depicted example, layer 510 comprises polydimethylsiloxane (PDMS); however, other materials, such as metals, polymers, glass, ceramics, and the like can be used for layer 510 without departing from the scope of the present invention.

FIG. 5B depicts nascent feature layer 512 resident in mold 500.

At operation 403, layer 510 is cured to convert nascent feature layer 512 into feature layer 308.

At operation 404, feature layer 308 is removed from the substrate, typically by peeling it out of mold 500.

FIG. 5C depicts feature layer 308 after its removal from mold 504.

Once removed from mold 500, feature layer 308 is wrapped around mandrel 306 to complete the fabrication of form 302. Feature layer is wrapped around mandrel such that surface 324 is placed in contact with surface 318 of mandrel 306.

FIGS. 3C-D depict cross-sections of a portion of form 302 before and after, respectively, feature layer 308 is wrapped around mandrel 306. Once the mandrel and feature layer are in contact, surface 318 of mandrel 306 seals wells 320 to define a closed chamber having the shape of cap 118. Channels 322 define stalks 116, as well as provide access for liquid latex into wells 320.

In some embodiments, form 302 is another mold form suitable for defining the desired shape of layer 100. For example, in some embodiments, form 302 is a cylindrical glass rod having the approximate shape and dimensions desired for the finished product. In some embodiments, form 302 comprises photosensitive glass that is patterned to define surface features 310 using patterned light and then etched. In some embodiments, form 302 comprises another suitable material, such as silicon, photodefinable epoxy-based resins (e.g., SU-8), glass, Teflon, metal (e.g., steel, stainless steel, etc.), and the like.

In some embodiments, form 302 is formed by methods other than those described above, such as wrapping one or more wires around mandrel 306, 3D printing, injection molding, machining (e.g., sawing, cutting, laser cutting, drilling, laser ablation, etching, electrical-discharge machining, etc.) grooves into the surface of mandrel 306, and the like.

FIGS. 3E and 3F depict schematic drawings of an alternative process for fabricating a dip-coating form, before and after the formation of surface features 310, respectively, in accordance with the present invention.

Nascent mold 328 includes mandrel 330, which has substantially smooth surface 332. In the depicted example, mandrel 330 comprises a photodefinable material, such as Fotoform glass, Foturan, SU-8, and the like. In some embodiments, mandrel 330 includes a central core of non-photodefinable material and an outer shell of photodefinable material.

In order to form surface features 310, surface 332 is exposed to patterned light signal 334. This substantially converts the exposed material into a different material that can be more easily etched in an appropriate etchant. One skilled in the art will recognize, after reading this Specification, that careful control of the exposure of mandrel 330 is required and, in some cases, multiple exposures of the mandrel at different energy levels must be performed.

Once properly exposed, the mandrel is etched in a suitable etchant that attacks the converted material at a faster rate than the unexposed material. This produces completed form 336, as shown in FIG. 3F.

Returning now to method 200, at operation 202, bath 304 is provided.

Bath 304 includes liquid 312, which is a flowable form of material 114. Typically, liquid 312 is filtered for particulates and includes a solvent used to establish its viscosity such that the flowable liquid will fill surface features 310, as well as set the thickness of the structural layer.

At operation 203, liquid 312 is sterilized and degassed to remove volatile components that could form voids and bubbles in layer 100 once cured. In some embodiments, liquid 312 is degassed by exposing it to a vacuum (300 milliTorr, preferably less than 15 milliTorr) to draw out volatile components. It should be noted that, after liquid 312 is degassed, it is preferably introduced to form 302 without significant delay to mitigate the reabsorption of gases into the liquid while it is exposed to atmosphere. In some embodiments, liquid 312 is periodically degassed to mitigate its reabsorption of gas. In some embodiments, liquid 312 is maintained in a vacuum environment during the formation of layer 100.

It should be further noted that, while liquid 312 is exposed to vacuum, its solvent, such as water, can evaporate, thereby changing its viscosity. In some embodiments, therefore, solvent is periodically added to bath 304 at a rate that substantially matches its evaporation rate.

It should also be noted that features 310 are extremely small and may not completely fill with liquid 312 when the form is inserted into bath 304. For example, in some cases, the surface tension of liquid 312 can prevent liquid 312 from filling or completely filling features 310. However, by providing the interior surface (i.e., surface 314) of the surface features with a suitable surface characteristic such that this surface of the features attracts the liquid, liquid 312 can be drawn into features 310.

At operation 204, surface 314 is provided a surface characteristic that enables liquid 312 to coat surface 314 and be drawn into features 310. In the depicted example, surface 314 is provided an attractive force between itself and liquid 312 such that the liquid will spread completely over the entire exposed surface of the form, thereby “wetting” the surface completely.

In the depicted example, form 302 is provided such that surface 314 is PDMS; therefore, surface 314 is hydrophobic. As a result, since latex is a polar, or aqueous, preparation when in liquid form, liquid 312 will not wet surface 314 such that it is drawn into features 310 unless the surface is treated to make it hydrophilic. In operation 204, surface 314 is coated with a layer of aluminum to convert it from hydrophobic to hydrophilic. In some embodiments, surface 314 is coated with a different hydrophilic material, such as alumina, etc.—preferably via a deposition method (e.g., atomic-layer deposition, etc.) that results in a uniform coating. One skilled in the art will recognize that adding a layer of hydrophilic material to the surface represents merely one example of a method for converting the surface characteristics of surface 314 to facilitate the filling of features 310 and that myriad alternative methods can be used without departing from the scope of the present invention. For example, in some embodiments a hydrophobic surface (e.g., PDMS, silicon, etc.) is treated with an oxygen plasma to convert to a hydrophilic surface. Alternative methods for converting a surface from hydrophobic to hydrophilic include, without limitation, chemically altering the surface, adding a coating, plasma surface treatments, etc.

In some embodiments, surface 314 is provided with the surface characteristic of a hydrophobic surface that is suitable for use with non-polar preparations of a desired material.

In some embodiments, form 302 includes features that are operative for allowing trapped air to escape from the layer of material 312 as it is disposed on the surface of the form.

At operation 205, form 302 is introduced into bath 304 to coat the form with a layer of liquid 312. Preferably, form 302 is introduced into liquid 312 at a controlled rate into the liquid to avoid formation of air bubbles that could otherwise be trapped in the mold. One skilled in the art will recognize that the controlled introduction of liquid 312 to form 302 enables the hydrophilic surface to draw the liquid into the mold while simultaneously displacing air. In some embodiments, form 302 is dipped into liquid 312 under vacuum to further mitigate bubble formation. Once form 302 is removed from bath 304, the form is coated with nascent layer 316 (as depicted in FIG. 3A). In some embodiments, the form is continuously rotated so as to cover any remaining uncovered portions of the mold.

At operation 206, nascent layer 316 is cured to form layer 100.

One skilled in the art will recognize, after reading this Specification, that the above-described method for forming projections 106 represents only one of myriad methods in accordance with the present invention. Methods suitable for forming projections on a surface in accordance with the present invention include, without limitation, selective deposition, selective etching, etching, lithography, injection molding, molding, abraiding, machining (e.g., drilling, cutting, computer-numerical control (CNC), etc.), laser-assisted etching, cutting (e.g., laser cutting, water cutting, sawing, etc.), sand blasting, roughening, 3D printing, laminating, plating, electrical-discharge machining (EDM), casting, imprinting, embossing, reel-to-reel transfer, including suitably sized/shaped particles in layer 314, and the like. In some embodiments, projections 106 are added to layer 100 by attaching them to at least one of its surfaces (e.g., via a layer transfer process, etc.). For example, in some embodiments, grooves are cut into a cylindrical mandrel having an appropriate initial diameter via a thin wire saw. The grooves can be formed by moving the wires relative to the mandrel or by spinning the mandrel while moving the wire saw into it.

At operation 207, layer 100 is removed from form 302. Preferably, layer 100 is carefully removed in a manner that mitigates the potential for a surface of the layer from sticking to itself. In some embodiments, removal is performed while the layer and form are submerged in a bath or held in an atmosphere that substantially prevents such sticking.

At optional operation 208, a surface treatment is applied to at least one of surfaces 108 and 110 to provide the surface with one or more desired surface characteristics, such as hydrophobicity, friction coefficients, and the prevention of self-sticking. Methods suitable for treating the surface of layer 100 include, without limitation, exposing the surface to air, applying an anti-sticking coating, and the like.

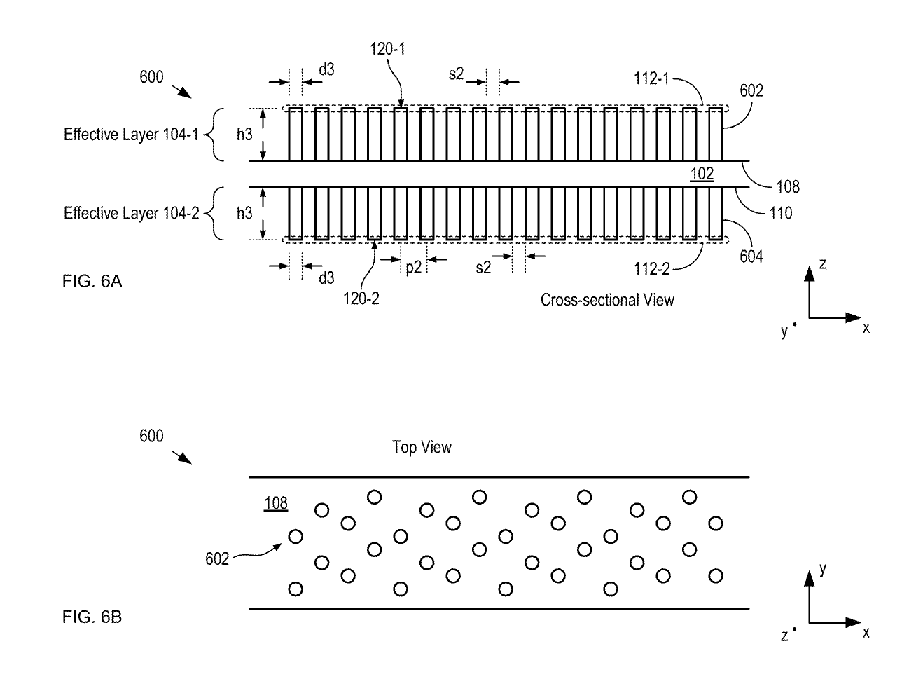

FIGS. 6A-B depict schematic drawings of cross-sectional and top views, respectively, of a portion of a sheath layer having engineered feels on both sides in accordance with a first alternative embodiment of the present invention. Layer 600 is analogous to layer 100; however, layer 600 includes an effective layer on both sides of base layer 102 (i.e., base layer 102 is located between effective layers 104-1 and 104-2). Effective layers 104-1 and 104-2 have effective surfaces 112-1 and 112-2, respectively. In the depicted example, layer 600 is dimensioned and arranged to define a condom that provides a first desired feel to the condom user and a second desired feel to the partner of the user.

Each of effective layers 104-1 and 104-2 is analogous to effective layer 104 described above and with respect to FIGS. 1A-E; however, in the depicted example, each of effective layers 104-1 and 104-2 includes a plurality of stand-alone columns as opposed to walls.

Projections 602 and 604 are analogous to projections 106; however, projections 602 and 604 are substantially identical stand-alone individual columns having a high aspect ratio. Preferably, the aspect ratio of each of projections 602 and 604 is within the range of approximately 2:1 to approximately 40:1. Projections 602 and 604 have substantially uniform diameter, d3, of 100 microns and height, h3, of 800 microns. In some embodiments, d3 is another diameter within the range of approximately 10 nanometers to approximately 1 millimeter. In some embodiments, h3 is another height within the range of approximately 10 nanometers to a few centimeters.

Preferably, projections 602 and 604 are high-contrast projections, as discussed above.

Projections 602 and 604 are arranged in a two-dimensional ordered arrays having periodicity that gives rise to a uniform spacing, s2, between the projections of 500 microns. In some embodiments, the spacing between projections is another spacing within the range of approximately 10 nanometers to approximately 1 millimeter; however, other spacings can be used without departing from the scope of the present invention. In some embodiments, projections 602 and/or 604 are arranged in an arrangement that is aperiodic in at least one dimension. In such embodiments, s2 refers to the average spacing between projections, where the maximum spacing within the arrangement remains less than or equal to 1 millimeter. In some embodiments, the projections of at least one of effective layers 104-1 and 104-2 are arranged in an ordered array having a spacing that is uniform in two dimensions.

In some embodiments, at least one of effective layers 104-1 and 104-2 has one or more projections having a shape, aspect ratio, diameter, height, and/or arrangement that is different than the projections of the other effective layer. Such embodiments enable layer 600 to transmit a different sensory characteristic on either side of layer 600. For example, in some embodiments, projections 602 are dimensioned and arranged to provide the feel of a first material while projections 604 are dimensioned and arranged to provide the feel of a different, second material. In the case of some condom embodiments, for example, projections 602 are designed to simultaneously transmit the feel of vaginal tissue to the condom wearer while projections 604 are designed to transmit the feel of penile/erectile tissue to the wearer's partner.

In some embodiments, the projections of at least one of effective layers 104-1 and 104-2 include projections that are arranged in a two-dimensional arrangement within at least one field, wherein the projections are arranged such that they have a fill factor within the field that facilitates interaction with tissue or other material with which the projections come into contact. In some embodiments, the projection fill-factor of an effective layer is within the range of approximately 0.0001% to approximately 99%. In some embodiments, the projection fill-factor is within the range of approximately 0.04% to approximately 72%. In the illustrative embodiment described herein, each of effective layers 104-1 and 104-2 have a projection fill-factor within the range of approximately 2% to 20%, which provides the tactile characteristics of velvet. In some embodiments, a surface includes projections arranged with a projection fill-factor that provides the tactile characteristics of a different surface, such as vaginal tissue, mucous membranes, anal tissue, human skin, cancerous tissue, healthy tissue, chicken skin, lizard skin, a phonograph record, corduroy, fur, and the like. In some embodiments, at least one of effective layers 104-1 and 104-2 includes projections that are operative for enhancing the feel of a desired surface to the user and/or the partner of the user.

It should be noted that, although projections 602 and 604 are columns, in some embodiments, at least one of projections 602 and 604 is a different type of projection, such as a wall, block, etc., as described above and with respect to FIGS. 1A-E, without departing from the scope of the present invention. Further, although projections 602 and 604 are depicted as being aligned with one another in the z-direction, in some embodiments, projections 602 and 604 are not aligned with one another.

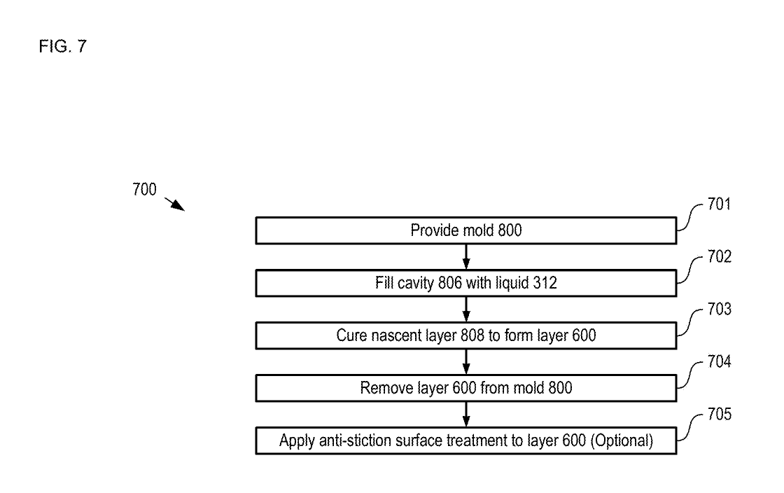

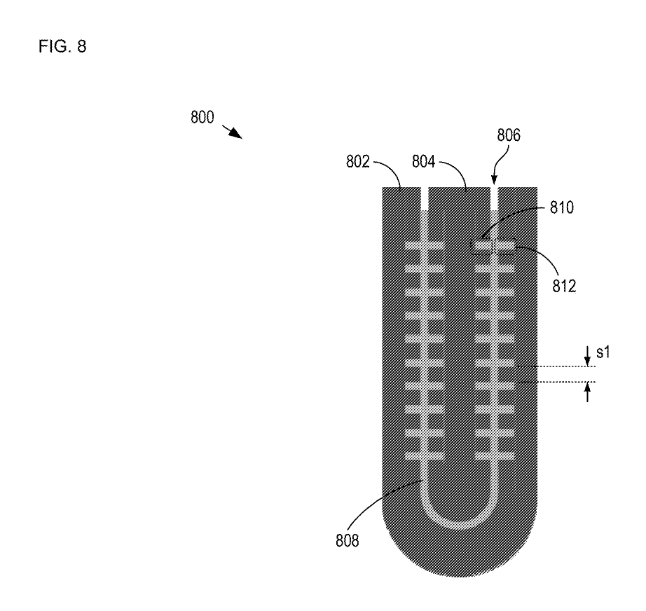

FIG. 7 depicts operations of a method suitable for forming layer 600 as a condom. Method 700 is a slightly modified version of method 200 described above and begins with operation 701, wherein a mold for layer 600 is provided. Method 700 is described herein with continuing reference to FIGS. 6A-B and reference to FIG. 8.

FIG. 8 depicts a schematic drawing of a mold suitable for templating layer 600 as a condom. FIG. 6 depicts the formation of layer 600 while the layer is in its nascent state. Mold 800 is a mold suitable for injection molding and includes shell 802 and core 804. One skilled in the art will recognize, after reading this Specification, that method 700 and mold 800 represent only one of many ways in which layer 600 can be formed without departing from the scope of the present invention.

Shell 802 is a conventional “clam-shell” mold having two halves that can be assembled to define a single reservoir.

Core 804 is analogous to form 302 described above and includes surface features 810, which are analogous to surface features 310.

Core 804 is inserted into and held within the reservoir of shell 802 to define cavity 806, which has the approximate shape and dimensions desired for a condom having a plurality of features projecting from its inner and outer surfaces, respectively. Shell 802 includes a plurality of surface features 812, which are also analogous to surface features 310. Surface features 810 and 812 are dimensioned and arranged to form projections 602 and 604. In the first alternative embodiment, mold 800 has surface characteristics suitable for drawing latex, in flowable form, into features 810 and 812. For example, in some embodiments, mold 800 is provided such that it has a hydrophilic surface suitable for use with polar or aqueous preparations of a desired material. In some embodiments, mold 800 has a hydrophobic surface suitable for use with non-polar preparations of a desired material. In some embodiments, mold 800 includes features that are operative for allowing trapped air to escape from a material disposed on the surface of the form.

At operation 702, cavity 806 is filled with liquid 312. Typically, liquid 312 is forced into cavity 806 using force or pressure. In some embodiments, cavity 806 is evacuated of air before liquid 312 is injected. In some embodiments, liquid 312 is drawn into cavity 806 using vacuum.

At operation 703, nascent layer 808 is cured to form layer 600.

At operation 704, layer 600 is removed from mold 800. Typically, layer 600 is removed from the mold by disassembling shell 802 and removing layer 600 from core 804, as described above; however, one skilled in the art will recognize that this is only one of myriad ways in which the layer can be removed from the mold. As discussed above, preferably, layer 600 is carefully removed in a manner that mitigates the potential for a surface of the layer from sticking to itself. In some embodiments, removal is performed while the layer and form are submerged in a bath or held in an atmosphere that substantially prevents such sticking.

At optional operation 705, a surface treatment is applied to layer 600 modify its surface to prevent self-sticking. Methods suitable for treating the surface of layer 600 include, without limitation, exposing the surface to air, applying an anti-sticking coating, and the like.

In some embodiments, either of layers 100 and 600 includes a plurality of projection types (i.e., projections having shapes other than the simple columns of projections 602 and 604).

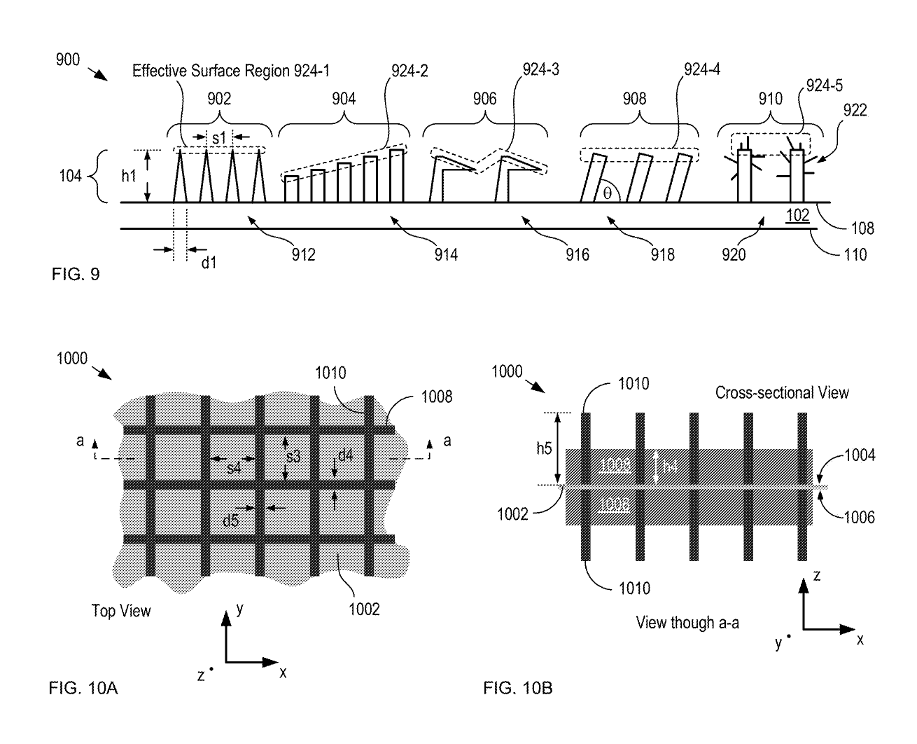

FIG. 9 depicts a schematic drawing of a cross-sectional view of a layer having improved sensory characteristics in accordance with a second alternative embodiment of the present invention. Layer 900 includes fields 902, 904, 906, 908, and 910, each of which selectively includes a different projection type. Each of fields 902, 904, 906, 908, and 910 includes a plurality of projections whose free ends (i.e., the ends distal to base layer 102) define an effective surface that is designed to provide a different sensory transmission characteristic. Specifically, fields 902, 904, 906, 908, and 910 define effective surface regions 924-1 through 924-5, respectively.

Field 902 comprises a plurality of projections 912. Each of projections 912 is a projection type having a conical shape. Projections 912 have substantially uniform height, h1, and a diameter that changes from d1 at its base (i.e., where it meets surface 108) to a sharp point at its tip. Projections 912 are substantially uniformly spaced such that their tips are separated by spacing, s1. In some embodiments, field 902 includes at least one conically shaped projection having a different diameter and/or height and/or stiffness, or that is spaced from at least one nearest neighbor by a different spacing. In some embodiments, at least one conically shaped projection included in field 902 terminates at a flat top surface rather than a point.

Field 904 comprises a plurality of projections 914. Each of projections 914 is a projection type having a cylindrical shape. Within field 904, projections 914 have different heights and substantially uniform diameter, d1. In some embodiments, field 904 includes at least one cylindrically shaped projection having a diameter different than d1. In some embodiments, field 904 includes cylindrically shaped projections that are the same height, but at least one projection has a diameter other than d1. In some embodiments, field 904 includes a plurality of cylindrically shaped projections, wherein at least one of the projections has a different stiffness.

Field 906 comprises a plurality of projections 916. Each of projections 916 is a projection type having a curved shape. Within field 906, projections 916 have different heights, lengths, and diameters; however, their effective width and spacing at their free end remains at or below one millimeter. In some embodiments, field 906 includes curved projections wherein at least one of their heights, lengths, and diameters is substantially uniform across the field. In some embodiments, field 906 includes a plurality of curved projections, wherein at least one of the projections has a different stiffness.

Field 908 comprises a plurality of projections 918. Each of projections 918 is a projection type having a cylindrical shape, where the cylinder extends from surface 108 at angle, θ, which is a non-normal angle (i.e., the projection does not form 90° angle with the surface).

Field 910 comprises a plurality of projections 920. Each of projections 920 is a projection type having a cylindrical shape. Each of projections 920 includes one or more sub-projections 922 that extend from one or more surfaces of the projection (i.e., its top or side surfaces). In some embodiments, within field 910, at least one of projections 920 has at least one of a different height, different diameter, and different shape.

In some embodiments, one or more of fields 902, 904, 906, 908, and 910 is formed on surface 110 of layer 900. In some embodiments, one or more of fields 902, 904, 906, 908, and 910 is formed on both of surfaces 108 and 110 of layer 900. In some embodiments, a field includes at least one wall, as discussed above and with respect to FIGS. 1A-E and/or one support line and/or sensation line as described below and with respect to FIGS. 10A-B.

In some embodiments, at least one of surfaces 108 and 110 includes a mixture of different projection types within a single field. In other words, in some embodiments, different projection types are intermingled on a surface of a layer. In some of these embodiments, the different projection types are intermingled across an entire surface (i.e., the surface has only one field, which includes a mixture of projection types and/or support/sensation lines).

In some embodiments, the projections within an individual field and/or across multiple fields are non-uniform. In other words, at least one projection in one field differs from at least one other projection in the same field and/or a different field in height, width, aspect ratio, diameter, cross-sectional shape, and/or angle, θ.

FIGS. 10A-B depict schematic drawings of top and cross-sectional views, respectively, of a layer having improved sensory characteristics in accordance with a third alternative embodiment of the present invention. Layer 1000 includes membrane 1002, support lines 1008 and sensation lines 1010. Layer 1000 is analogous to layer 100 described above and with respect to FIGS. 1A-E. The cross-sectional view shown in FIG. 10B is taken through line a-a shown in FIG. 10A.

Membrane 1002 is a layer of latex having a thickness of 50 microns. In some embodiments, membrane 1002 has another thickness within the range of approximately 10 microns to approximately 200 microns and, preferably, less than or equal to 100 microns. Membrane 1002 has top surface 1004 and bottom surface 1006.

Support lines 1008 project outward from each of top surface 1004 and bottom surface 1006 and are linear along the x-direction, as indicated. Each of support lines 1008 has a width, d4, of 100 microns and height, h4, of 400 microns. Preferably, d4 is within the range of approximately 10 microns to approximately 1 millimeter and, typically, within the range of approximately 10 microns to approximately 500 microns. Preferably, height, h4, is within the range of approximately 10 microns to approximately 5000 microns. Support lines 1008 are spaced apart from one another by a spacing, s3, of 500 microns. Typically, s3 is within the range of approximately 100 microns to approximately 1 millimeter. It should be noted that, in embodiments wherein support lines 1008 do not significantly contribute to the feel of layer 1000, each of d4 and s3 can have any practical and can exceed 1 millimeter. In some embodiments, such as layer 100 described above, support lines 1008 are not included.

Sensation lines 1010 project outward from each of top surface 1004 and bottom surface 1006 and are linear along the y-direction, as depicted. Each of sensation lines 1010 has a width, d5, of 100 microns and height, h5, of 800 microns. In some embodiments, d5 is another width within the range of approximately 10 microns to approximately one millimeter, and, preferably, within the range of approximately 10 microns to approximately 500 microns. In some embodiments, h5 is another height within the range of approximately 10 microns to approximately 5000 microns.

Sensation lines 1010 are spaced apart by a spacing, s4, of 500 microns. In some embodiments, s4 is another spacing within the range of approximately 100 microns to approximately 1 millimeter.

In some embodiments, particularly those that do not include support structure, such as support lines 1008, sensation lines 1010 are formed such that they are substantially self-supporting. For example, in some embodiments, sensation lines 1010 have a wider base than top. In some embodiments, sensation lines 1010 have a width that mitigates their touching each other during formation and/or until fully cured.

Although support lines 1008 and sensation lines 1010 are depicted as being straight in FIGS. 10A-B, in some embodiments, at least one of support lines 1008 and sensation lines 1010 has a non-linear shape, such as curved, wavy, polygonal, etc. Further, in some embodiments, at least one of support lines 1008 and sensation lines 1010 has a different width, height, and/or spacing.

One skilled in the art will recognize that the projection types disclosed herein represent only a few of myriad projection types that are within the scope of the present invention. Other projection types suitable for use in embodiments of the present invention include, without limitation, ridges, partial ridges, lines, curves, waves, star shapes, octagons, irregularly shaped projections, projections having sub-features that project in any direction, etc.

In some embodiments, the surface characteristics of a layer, such as hydrophobicity, hydrophilicity, friction, stiction, liquid contact angle, and surface energy, are controlled by applying a surface treatment to the surface and the projections that extend from the surface. In some embodiments, the surfaces of a layer are provided different surface coatings such that each surface has different surface characteristics. In some embodiments, one surface of a layer is left uncoated. Surface treatments for controlling a surface characteristic in accordance with the present invention include, without limitation, roughening treatments (e.g., plasma etches, chemical etches, etc.), smoothing treatments (such as plasma etches, chemical etches, and he like), self-assembled monolayers (e.g., octadecyltrichlorosilane, fluoro-octo-trichlorosilane (FOTS), perfluorodecyltrichlorosilane (FTDS), etc.), chemical treatments, vapor and liquid phase depositions, atomic layer deposition, and the like. In some embodiments, a surface and its associated projections are treated with a surface treatment that includes nanostructures, such as nanoparticles, carbon nanotubes, etc.

It is to be understood that the disclosure teaches just a few examples of embodiments of the present invention and that many variations of the invention can easily be devised by those skilled in the art after reading this disclosure and that the scope of the present invention is to be determined by the following claims.

1. A condom comprising a layer (100) of a first material (114) characterized by a bulk modulus of elasticity, λ1, the layer including:
a base layer (102) having a first surface (108) and a second surface (110); and
a first plurality of projections (106) that project from the first surface, the first plurality of projections collectively defining a first effective layer (104), wherein each projection of the first plurality thereof includes a first end face (120) and a first stalk (116) having a first aspect ratio, and wherein the first end faces of the first plurality of projections collectively define a first effective surface (112);
wherein the first effective layer is characterized by a first effective modulus of elasticity, λ2, that is based on the first aspect ratio;
wherein each first end face has a first width (d2) in at least one dimension, and wherein the first plurality of projections is arranged in a first arrangement in which the first end faces are spaced apart by a first spacing (s1) along the at least one dimension, and further wherein each of the first width and first spacing is less than or equal to 1 mm; and
wherein λ2 is lower than λ1.

2. The condom of claim 1 wherein at least one of the first aspect ratio, first width, and first spacing of the first plurality of projections is non-uniform.

3. The condom of claim 1 wherein the first arrangement is periodic in the at least one dimension.

4. The condom of claim 1 wherein the first aspect ratio is within the range of approximately 2:1 to approximately 40:1.

5. (canceled)

6. The condom of claim 1 wherein the first plurality of projections is arranged in a first arrangement that includes a first field having a fill-factor within the range of approximately 0.04% to approximately 72%.

7. The condom of claim 1 wherein the first plurality of projections includes a first plurality of projection types.

8. The condom of claim 7 wherein the projection types of the first plurality thereof are intermixed within the first arrangement.

9. The condom of claim 1 wherein a first projection of the first plurality of projections comprises a first cap (118) that includes the first end face of the first projection, wherein the first stalk of the first projection has a first cross-sectional area and the first cap has a second cross-sectional area that is equal to or greater than the first cross-sectional area.

10. (canceled)

11. The condom of claim 1 wherein a first projection of the first plurality of projections comprises a first cap (118) that includes the first end face of the first projection, wherein the first stalk of the first projection has a first cross-sectional area and the first cap has a second cross-sectional area that is smaller than the first cross-sectional area.

12. The condom of claim 1 further including a second plurality of projections (604) that project from the second surface (110), the second plurality of projections collectively defining a second effective layer (104-2), wherein each projection of the second plurality thereof includes a second end face (120-2) and a second stalk having a second aspect ratio, and wherein the second end faces of the second plurality of projections collectively define a second effective surface (112-2);
wherein the second effective layer is characterized by a second effective modulus of elasticity, λ3, that is based on the second aspect ratio;
wherein each second end face has a second width (d3) in at least one dimension, and wherein the second plurality of projections is arranged in a second arrangement in which the second end faces are spaced apart by a second spacing (s2) along the at least one dimension, and further wherein each of the second width and second spacing is less than or equal to 1 mm; and
wherein λ3 is lower than λ1.

13. The condom of claim 12 wherein λ3 and λ2 are equal.

14. The condom of claim 1 wherein at least one of the first plurality of projections is a high-contrast projection.

15. The condom of claim 1 wherein the first plurality of projections is operative for holding a first fluid.

16. The condom of claim 1 wherein the first effective layer includes a second plurality of projections that project from the first surface, the first plurality of projections being located within a first field (902) and the second plurality of projections being located in a second field (904), and wherein the first field selectively includes a first projection type (912) and the second field selectively includes a second projection type (914).

17. The condom of claim 1 wherein a first projection (920) of the first plurality of projections (910) includes a first sub-projection (922) that projects from a surface of the first projection.

18. A method for forming a condom having an engineered feel on at least one surface, the method comprising:
(1) providing a first layer (100) such that it defines the condom sheath, wherein the first layer is provided such that it is homogeneous and comprises a first material (114) that is characterized by a bulk modulus of elasticity, λ1, the first layer comprising:
(a) a base layer (102); and
(b) a first effective layer (104) having a first effective modulus of elasticity, λ2, wherein the first effective layer includes a first effective surface (112) having a first fine roughness;
(2) providing the first effective layer as a first plurality of projections (106) that project from a first surface (108) of the base layer, each projection of the first plurality thereof including:
(a) a first stalk (116), wherein the first stalk is characterized by a first aspect ratio; and
(b) a first cap (118) having a first end face (120) that has a first width (d2) in at least one dimension;
wherein the first plurality of projections is arranged in a first arrangement in which the first end faces are spaced apart by a first spacing (s1) along the at least one dimension;
(3) selecting each of the first width and the first spacing such that it is less than or equal to 1 mm, wherein the first fine roughness is based on the first width and the first spacing; and
(4) selecting the first aspect ratio such that λ2 is lower than λ1, wherein λ2 is based on the first aspect ratio.

19. The method of claim 18 wherein the first layer is formed by operations further comprising:
providing a form (302) having a first surface (314) that includes a first plurality of surface features (310);
coating the form with a first liquid (312) that comprises the first material;
curing the first liquid; and
removing the cured first liquid from the form.

20. The method of claim 19 wherein the first layer is formed by operations further comprising providing the first surface with a surface characteristic that facilitates the drawing of the a first liquid into each of the plurality of surface features.

21. The method of claim 19 wherein the form is provided by operations comprising:
providing a mandrel (306) having a first surface (318);
forming a feature layer (308) such that it comprises the first plurality of surface features and a second surface (324); and
wrapping the feature layer around the mandrel such that the first surface and second surface are in contact.

22. The method of claim 19 wherein the form is provided by operations comprising:
providing a mandrel (330) having a second surface (332); and
forming the first plurality of surface features at the second surface.

23. The method of claim 22 wherein the first plurality of surface features are formed by operations comprising:
providing the mandrel such that it comprises a second material that is photodefinable such that exposure to light having a first wavelength converts the second material into a third material;
exposing the second surface to a patterned light signal (334), the patterned light signal comprising the first wavelength; and
etching the mandrel in a first etchant that etches the third material at a faster rate than the second material.

24. The method of claim 22 wherein the first plurality of surface features are formed by machining the second surface via a process selected from the group consisting of three-dimensional (3D) printing, sawing, wire sawing, drilling, and milling.

25. (canceled)

26. The method of claim 21 further comprising:
(5) encasing the form in a mold cavity (802), the mold cavity comprising a second plurality of surface features (812) that are arranged in a second arrangement in a second field such that the spacing between the surface features of the second plurality thereof are spaced apart by a spacing, s1, that is less than or equal to one millimeter; and
removing the form from the mold cavity after the first liquid is cured.

27. The method of claim 18 further comprising:
(5) providing a second effective layer (104-2) that includes a second plurality of projections (604) that project from a second surface (110) of the base layer, wherein the first layer includes the second effective layer, and wherein the second effective layer has a second effective modulus of elasticity, λ3, and a second effective surface (112-2) having a second fine roughness, and further wherein each projection of the second plurality thereof includes:
(a) a second stalk (116), wherein the first stalk is characterized by a second aspect ratio; and
(b) a second cap (118) having a second end face (120) that has a second width (d3) in at least one dimension;
wherein the second plurality of projections is arranged in a second arrangement in which the second end faces are spaced apart by a second spacing (s2) along the at least one dimension;
(6) selecting each of the second width and the second spacing such that it is less than or equal to 1 mm, wherein the second fine roughness is based on the second width and the second spacing; and
(7) selecting the second aspect ratio such that λ3 is lower than λ1, wherein λ3 is based on the second aspect ratio.
